(19) United States
(12) Patent Application Publication
(10) Pub. No.: US 2022/0220299 A1
(43) Pub. Date: Jul. 14, 2022

(54) ANTIMICROBIAL MATERIALS BASED ON POLYLACTIC ACID OLIGOMERS, PROCESS FOR PREPARING THE SAME, AND USE THEREOF

(71) Applicant: The Hong Kong Polytechnic University, Hong Kong (CN)

(72) Inventors: Xiaoming Tao, Hong Kong (CN); Ziheng Zhang, Hong Kong (CN); Linlin Ma, Hong Kong (CN)

(73) Assignee: The Hong Kong Polytechnic University, Hong Kong (CN)

(21) Appl. No.: 17/569,743

(22) Filed: Jan. 6, 2022

Related U.S. Application Data
(60) Provisional application No. 63/135,814, filed on Jan. 11, 2021.

(51) Int. Cl. C08L 67/04; D06M 16/00

(52) U.S. Cl. CPC C08L 67/04; C08L 2203/02; D06M 16/00

(57) ABSTRACT

This disclosure provides a biodegradable antimicrobial material based on polylactic acid oligomer represented by formula 1 as an active ingredient. The disclosure also provides a process for preparing the polylactic acid oligomer and an antimicrobial product prepared therefrom.

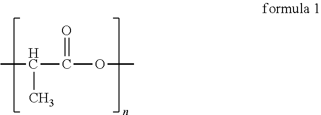

### CROSS REFERENCE TO RELATED APPLICATIONS

This application claims priority under 35 U.S.C. § 119(e) to U.S. Provisional Patent Application No. 63/135,814, filed Jan. 11, 2021. The foregoing application is incorporated by reference herein in its entirety.

### TECHNICAL FIELD

This application relates to the field of antibacterial materials, in particular to a polylactic acid oligomer-based antibacterial material and its production method and its application in such fields as textiles, medical treatment, and hygiene.

### BACKGROUND OF ART

Prevention of microbial transmission and infection has been a major public health and safety concern world-wide. Microbial infections not only cause diseases and wound pain, but also lead to the increase of morbidity and mortality in severe cases. Antimicrobial agents have been widely applied in agriculture, the environment, medicine, textiles, food, food package and other industry to protect people against the microorganisms. For antimicrobial materials, present technology includes inorganic, organic and natural type. Among the three types of commonly used antimicrobial agents, the inorganic ones exhibit high antimicrobial efficiency and good heat stability. However, there is an increasing concern on their potential perniciousness to environment and human health from the leaching out of heavy metals. Organic antimicrobial materials are commonly used, these agents include quaternary ammonium, polybiguanides, N-halamine materials and so on. However, their functional modification on the surface of materials shows poor durability. Antibiotics have been commonly used to prevent the bacteria infection, whereas the overuse of them lead to antibiotic resistance, which has become a worldwide threat to human health. The natural antimicrobial agents are safe, bioactive, nontoxic and environment-friendly, hence attract more and continuous attention. It has been found that many essential oils (Eos) were effective to kill MDR bacteria and can be applied with antibiotics for enhance the efficacy synergistically. However, the natural components of EOs cannot be controlled accurately, while the low durability and high volatility restrict their applications.

It is reported that poly (3-hydroxybutyrate) (S-PHB) oligomer has excellent antimicrobial effect and is degradable, durable, eco-friendly and safe, thus has significant advantages for the applications in healthcare field. In the Chinese patent CN110452121A, we report a bio-based material of poly (3-hydroxybutyrate) oligomer with antimicrobial properties. However, there is no report about antimicrobial property of polylactic acid oligomers so far.

### SUMMARY

In one aspect, the application provides a series of antimicrobial materials based on polylactic acid oligomers having antimicrobial property.

The antimicrobial materials based on polylactic acid (PLA) oligomers provided by this application contain oligomers represented by formula 1 as antimicrobial active ingredients,

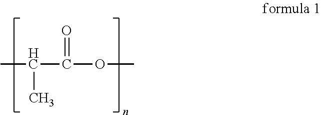

where n represents an integer of 1 to 20, for example 3 to 7.

The polylactic acid oligomers have number-average molecular weights of 100 to 2000, for example 200 to 600.

In another aspect, the application provides a process for preparing the polylactic acid oligomers as follows: at 100 to 300° C. (for example 180 to 230° C.), at the pressure of 5 Pa˜5 MPa (for example 1 kPa to 0.2 MPa), under nitrogen protection, stirring and heating monomer of lactic acid, and keeping the reaction of polymerization for 0.01˜72 hours (for example 1 to 8 hr).

The polylactic acid oligomer based antimicrobial material may contain any addition component, addition material, auxiliary material etc, except the active ingredient used in the antimicrobial material, provided that the active ingredient can be made into any form or any type of antimicrobial material without affecting the antimicrobial activity of the active ingredient, the application shall not be restricted.

In another aspect, the application provides use of the polylactic acid oligomer in the preparation of antimicrobial material.

In further aspect, the application provides an antimicrobial product obtained from the above mentioned antimicrobial material, including antimicrobial agents, antimicrobial fabrics, sanitary wares, and medical materials, etc., especially in the fields of textile, medical and health care. The antimicrobial material is applied to prepare one or more of antimicrobial preparations, antimicrobial fabrics, sanitary products, and medical materials. The antimicrobial material can be used to coat or modify existing polymer textiles for antimicrobial agents, drugs, disinfectants, medical materials, wound coating materials, dressings, surgical sutures, implant materials, drug carriers, and other biomedical materials. The antimicrobial materials involved in the application can be used separately or in combination with other antimicrobial materials, and can also be used to modify other antimicrobial materials.

Antimicrobial materials based on polylactic acid oligomers provided by the application have at least one of the following advantages:
(1) They have excellent, stable and durable antimicrobial activity and has excellent antimicrobial effect against many kinds of bacteria, fungi and viruses.
(2) In addition to having antimicrobial activity, the materials of the application also have excellent biodegradability, biocompatibility, low cost, high safety and environment-friendly properties.
(3) They can be made into various forms or types of antimicrobial materials or products, and can also be used in combination with other antimicrobial materials, or they can be used to modify other materials to obtain better performance, which greatly enrich the types and fields of antimicrobial materials.
(4) They are suitable for industrial production and large-scale use without complicated preparation process.

Items
1. An antimicrobial material based on polylactic acid oligomers, wherein the active ingredient of the antimicrobial material is polylactic acid oligomers represented by formula 1,

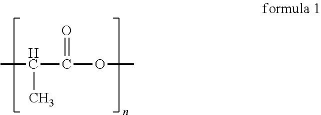

n represents an integer of 1 to 28.
2. The antimicrobial material according to item 1, wherein in the formula 1, n represents an integer of 3 to 7.
3. The antimicrobial material according to item 1 or 2, wherein the average molecular weight of the polylactic acid oligomer is 100 to 2000 g/mol.
4. The antimicrobial material according to any one of items 1 to 3, wherein the average molecular weight of the polylactic acid oligomer is 200 to 600 g/mol.
5. The antimicrobial material according to any one of items 1 to 4, wherein the antimicrobial material further contains other antimicrobial active ingredients.
6. The antimicrobial material according to any one of items 1 to 5, wherein the antimicrobial material is against a microbe selected from the group consisting of bacteria and virus.
7. A process for preparing the polylactic acid oligomer according to any one of items 1 to 6, wherein lactic acid monomers are stirred and heated under nitrogen atmosphere, and reacted for 0.01 to 72 hours at a temperature of 100 to 300° C. and a pressure of 5 Pa to 5 MPa.
8. The process according to item 7, wherein the temperature is 180 to 230° C.
9. The process according to item 7 or 8, wherein the pressure is 1 kPa to 0.2 MPa.
10. The process according to any one of items 7 to 9, wherein reaction is performed for 1 to 8 hrs.
11. An antimicrobial product prepared from the antimicrobial material according to any one of items 1-6.
12. The antimicrobial product according to item 11, which is selected from the group consisting of an antimicrobial preparation, an antimicrobial fabric, a sanitary product, and a medical material.

### DESCRIPTION OF DRAWINGS

FIG. 1 is liquid chromatography-mass spectrometry of the polylactic acid oligomer with a degree of polymerization of 3˜7.

### EMBODIMENTS

The following examples are for illustrative purposes only, and are not intended to limit the scope of protection of the present application.

### Example 1

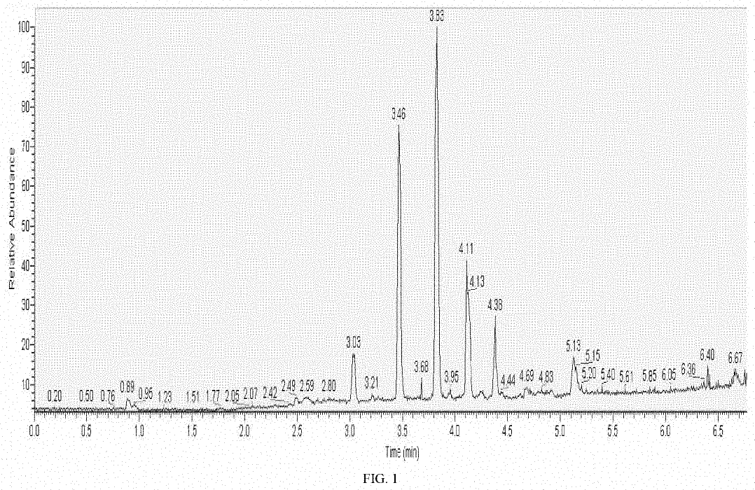

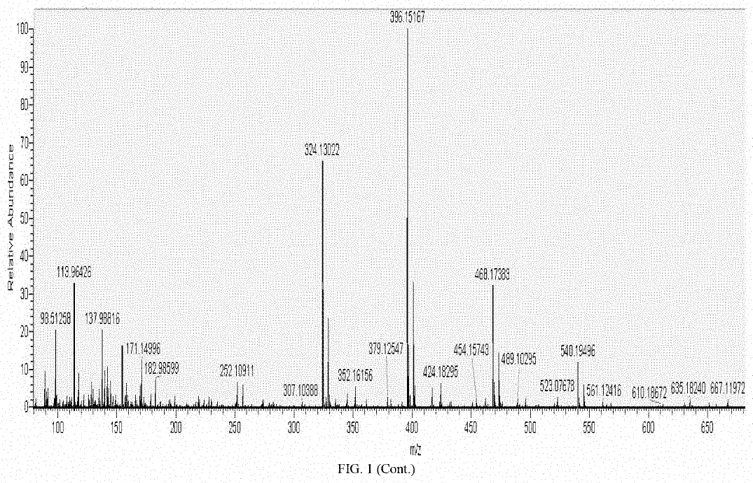

Adding 100 g of lactic acid to a reactor, under the protection of nitrogen, while stirring, the temperature was raised to 200° C. and maintained for 4 hours. The pressure in the reactor was maintained at one atmosphere. After the reaction, the temperature was lowered to room temperature, and all the reaction products were collected. A liquid chromatography-mass spectrogram was obtained by a mass spectrometer, as shown in FIG. 1. The degree of polymerization of the polylactic acid oligomer is about 4.

Property Testing

Diluting the polylactic acid oligomer obtained in Example 1 to 10 mg/mL or 20 mg/mL with phosphate buffer, and using the shaking method (referring to the national standard GB 15979-2002) to detect the antimicrobial activity, and PBS group as a blank control. The testing results are shown in Table 1.

TABLE 1
Antimicrobial performance of PLA oligomer

| | | | Concentration of | |
|---|---|---|---|---|
| | | | PLA oligomer | |
| Microbe | | | 10 | 20 |
| species | Test standard | Items | mg/ml | mg/ml |
| S. aureus | ASTM E2149 13a | Antimicrobial | >99.99 | >99.99 |
| (gram-positive | | reduction/% | | |
| bacteria) | | Antimicrobial | >4 | >4 |
| | | logarithm | | |
| | | value Mv | | |
| K. pneumoniae | ASTM E2149 13a | Antimicrobial | >99.99 | >99.99 |
| (gram-negative | | reduction/% | | |
| bacteria) | | Antimicrobial | >4 | >4 |
| | | logarithm | | |
| | | value Mv | | |
| E. coli | ASTM E2149 13a | Antimicrobial | >99.90 | — |
| (gram-negative | | reduction/% | | |
| bacteria) | | Antimicrobial | 3 | — |
| | | logarithm | | |
| | | value Mv | | |
| C. albicans | ASTM E2149 13a | Antimicrobial | 84.00 | 96.68 |
| (fungi) | | reduction/% | | |
| | | Antimicrobial | <1 | >1 |
| | | logarithm | | |
| | | value Mv | | |
| Methicillin- | ASTM E2149 13a | Antimicrobial | 80.73 | 84.44 |
| resistant | | reduction/% | | |
| Staphylococcus | | Antimicrobial | <1 | <1 |
| aureus | | logarithm | | |
| (multi-drug | | value Mv | | |
| resistant | | | | |
| bacteria) | | | | |
| Influenza A | Technical standard | Antimicrobial | — | >99.99 |
| virus subtype | for Disinfection | reduction/% | | |
| H1N1 | (2002, Ministry of | Antimicrobial | — | >4.00 |
| | Health P.R. China)- | logarithm | | |
| | 2.1.1.10.6, | value Mv | | |
| | 2.1.1.10.7. | | | |
| Influenza A | Technical standard | Antimicrobial | — | >99.99 |
| virus subtype | for Disinfection | reduction/% | | |
| H3N2 | (2002 Ministry of | Antimicrobial | — | >4.33 |
| | Health P.R. China)- | logarithm | | |
| | 2.1.1.10.6, | value Mv | | |
| | 2.1.1.10.7. | | | |

### Antimicrobial Finishing of Polypropylene Nonwoven Fabric

Liquid PLA oligomer was mixed with solvent (PLA oligomer: H₂O=1:10) to achieve homogenous solution, prior to be applied on the polypropylene nonwoven fabric through double-roll padding process. After evaporation of water by drying, the liquid PLA oligomer finished polypropylene nonwoven fabric was cooled down and collected in ambient condition for further antimicrobial test.

TABLE 2
Antimicrobial performance of PLA oligomer coated nonwoven
polypropylene fabrics

| Ratio of PLA | | | | | |
|---|---|---|---|---|---|
| oligomer | Testing | | | | Reduction |
| mass to | standard | | | | rate of |
| fabric | and | | | | microbes/ |
| mass % | method | Tested Microbe | Test time | | % * |
| 9.0 | ISO 18184: | Influenza A virus | 2 | h | >99.99 |
| | 2019 | subtype H3N2 | | | |
| 9.0 | ISO 18184: | Influenza A virus | 30 | min | >81.16 |
| | 2019 | subtype H3N2 | | | |
| 9.0 | ISO 18184: | Influenza A virus | 10 | min | >54.38 |
| | 2019 | subtype H3N2 | | | |
| 9.0 | ISO 18184: | Influenza A virus | 5 | min | >42.72 |
| | 2019 | subtype H3N2 | | | |
| 13.0 | ISO 18184: | Influenza A virus | 2 | h | >99.99 |
| | 2019 | subtype H3N2 | | | |
| 9.0 | ISO 18184: | Influenza A virus | 2 | h | >95.11 |
| | 2019 | subtype H1N1 | | | |
| 9.0 | ASTM | S. aureus | 1 | h | >99.99 |
| | E2149W, 2020 | | | | |
| 9.0 | ASTM E2149 | K. pneumoniae | 1 | h | >99.99 |
| | 20 | | | | |
| 9.0 | ASTM E2149 | C. albicans | 24 | h | 81.7% |
| | 20 | | | | |

* Difference between microbes in samples before and after the disinfectant action divided by microbes in samples before the disinfectant action. Reduction rate = (A-B)/A. A is microbes in samples before the disinfectant action. B is microbes in samples after the disinfectant action.

It can be seen from Tables 1 and 2 that the polylactic acid oligomer has excellent antimicrobial activity against microbes including bacteria and virus. Therefore, the degradable oligomer of the present application has extremely high application potential in preparing antimicrobial materials (especially medical antimicrobial materials) and antimicrobial product.

1. An antimicrobial material comprising a polylactic acid oligomer as an active ingredient, wherein the polylactic acid oligomer is represented by formula 1, 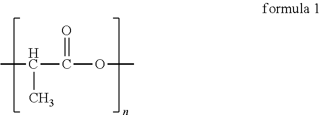
n represents an integer of 1 to 28.

2. The antimicrobial material according to claim 1, wherein in formula 1, n represents an integer of 3 to 7.

3. The antimicrobial material according to claim 1, wherein the average molecular weight of the polylactic acid oligomer is 100 to 2000 g/mol.

4. The antimicrobial material according to claim 1, wherein the number average molecular weight of the polylactic acid oligomer is 200 to 600.

5. The antimicrobial material according to claim 1, wherein the antimicrobial material further comprises one or more additional antimicrobial active ingredients.

6. The antimicrobial material according to claim 1, wherein the antimicrobial material is against a microbe selected from the group consisting of bacteria and virus.

7. A process for preparing the polylactic acid oligomer according to claim 1, wherein lactic acid monomers are stirred and heated under nitrogen atmosphere, and reacted for 0.01 to 72 hours at a temperature of 100 to 300° C. and a pressure of 5 Pa to 5 MPa.

8. The process according to claim 7, wherein the temperature is 180 to 230° C.

9. The process according to claim 7, wherein the pressure is 1 kPa to 0.2 MPa.

10. The process according to claim 7, wherein the reaction is performed for 1 to 8 hrs.

11. An antimicrobial product prepared from the antimicrobial material according to claim 1.

12. The antimicrobial product according to claim 11, which is selected from the group consisting of an antimicrobial preparation, an antimicrobial fabric, a sanitary product, and a medical material.
